(19) United States
(12) Patent Application Publication
(10) Pub. No.: US 2009/0171470 A1
(43) Pub. Date: Jul. 2, 2009

(54) Knee joint prosthesis

(76) Inventors: Hardy Bisinger, Rosenfled (DE); Gisela Fitzlaff, Vs-Schwenningen (DE); Volker Marek, Vs-Schwenningen (DE); Bernhard Holtkamp, Donaueschingen (DE); Gerhard Fitzlaff, Vs-Schwenningen (DE)

Correspondence Address: PYLE & PIONTEK, LLC, 221 N LASALLE STREET , ROOM 1207, CHICAGO, IL, 60601, US

(21) Appl. No.: 11/661,845

(22) PCT Filed: Apr. 26, 2005
(86) PCT No.: PCT/DE2005/000764

§ 371 Date: Apr. 27, 2008

(30) Foreign Application Priority Data
Apr. 30, 2004 (DE) 10 2004 021 250.3

(51) Int. Cl. A61F 2/64

(52) U.S. Cl. 623/43; 623/39

(57) ABSTRACT

A knee joint for a leg prosthesis comprising an upper joint part (2) and a lower joint part (3) which can be rotated about a joint axis (5) relative to the upper part of the joint (2), and an upper brake device (14, 15, 20, 21). The brake device comprises a first force transmission element 14, upon which the force triggering the brake effect acts, and which transfers the force at a given transmission ratio to a second force transmission element (15), which, in relation to the joint axis, transfers the displaced force in a substantially parallel direction towards a brake disc (20, 21).

The invention relates to a knee joint prosthesis according to the preamble of patent claim 1.

A knee joint prosthesis for a leg prosthesis is known from AT 3241695 C2 for example. The knee joint prosthesis has an upper and a lower joint part that are joined together through a joint axis. To produce a braking effect preventing the knee joint from bending under the body weight of the prosthesis wearer upon loading the leg prosthesis, there are provided two braking faces that are provided with a brake pad and produce a braking effect when loaded with weight by being caused to tilt toward each other. Such a knee joint prosthesis however does not allow for precise activation of the brake device through the magnitude of the body force acting thereon. Moreover, the brake is actuated, irrespective of the phase in which the leg prosthesis is when doing a step, i.e., the magnitude of the braking effect occurs independent of whether the point or the heel of the foot of the leg prosthesis is loaded. Accordingly, it is impossible to support natural ambulation.

Knee joint prostheses with what has been termed a winder brake are further known in which the radius of a slotted cylinder disposed coaxially to the joint axis is tapered so that the inner side of this cylinder acts onto the joint axis, thus producing a braking effect. The problem with this winder brakes however is that the brake does not immediately allow bending when unloaded, meaning that a self-locking effect occurs. This effect is the reason why the prosthesis wearers adopt an unnatural gait, causing them to always additionally lift the hip when moving it forward in the course of a step in order to release the winder brake.

It is the object of the invention to provide an improved knee joint prosthesis.

This object is solved by a knee joint prosthesis according to claim 1. Improved implementations of the invention are characterized in the dependent claims.

The particular advantage of the knee joint prosthesis is that the blocked brake device is immediately released when unloaded while the braking force achieved is load-dependent and proportional to the weight force introduced.

Further features and utilities of the invention will become apparent from the description of exemplary embodiments given with reference to the accompanying drawings. In the drawings:

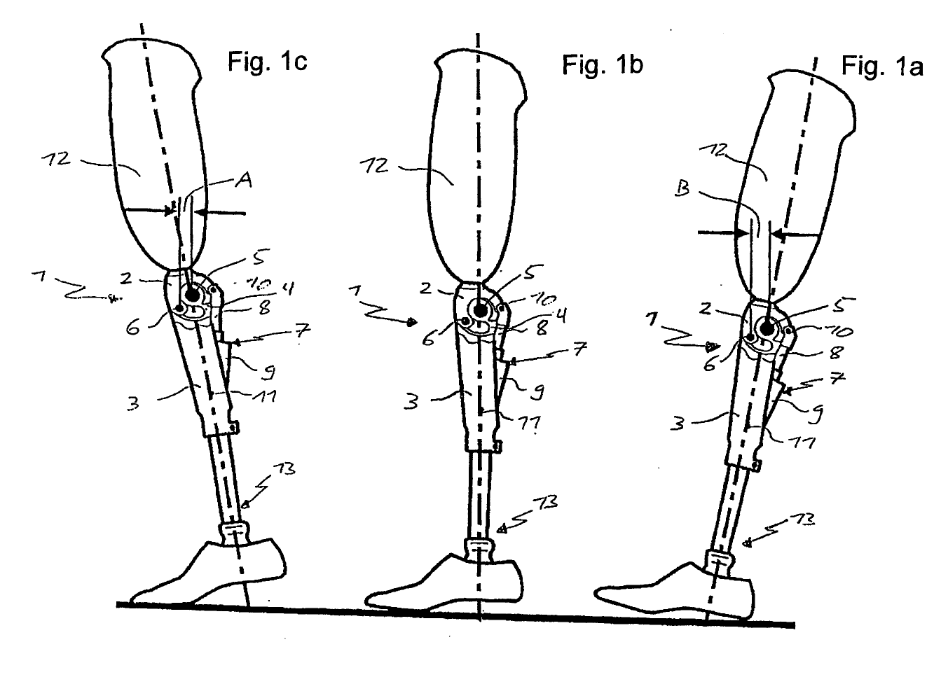

FIGS. 1a through 1c show a schematic illustration of a leg prosthesis with a knee joint of an embodiment of the present invention in three different leg positions;

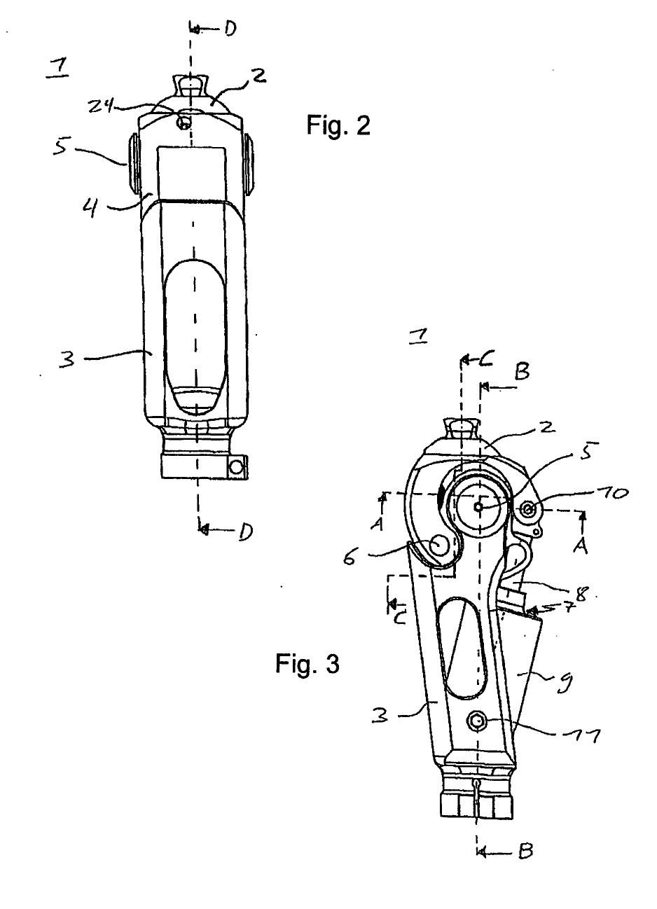

FIG. 2 is a front view of a knee joint of an embodiment of the present invention in the extended position;

FIG. 3 is a side view of the knee joint shown in FIG. 2;

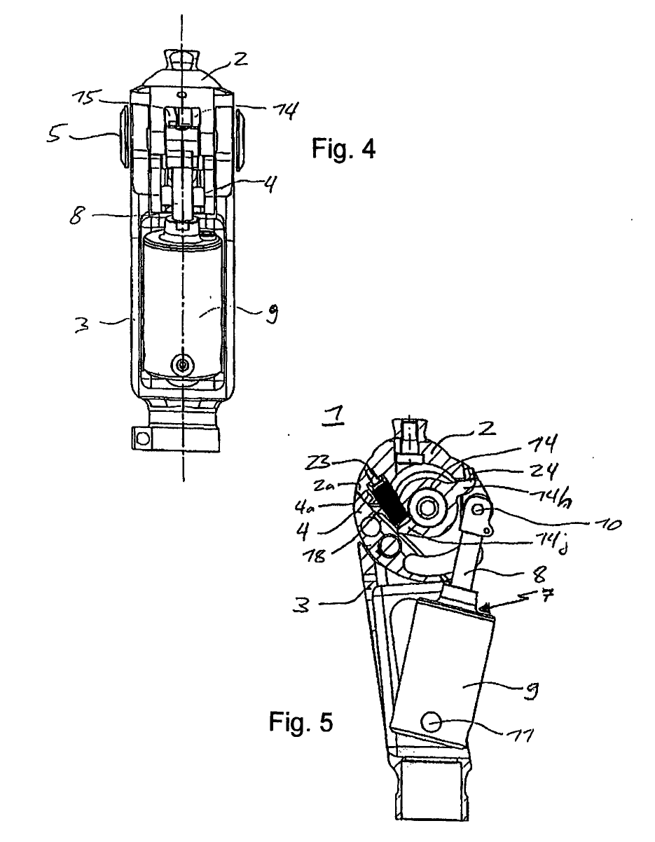

FIG. 4 is a rear view of the knee joint shown in FIG. 2;

FIG. 5 is a sectional side view of the knee joint shown in FIG. 2;

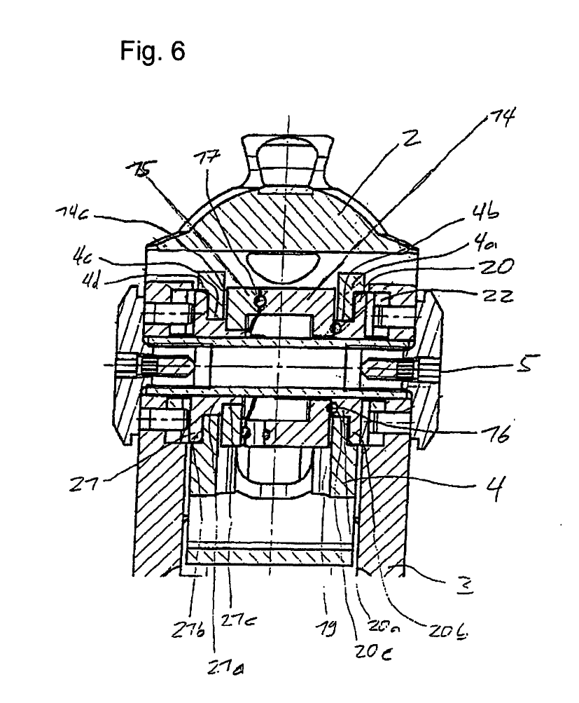

FIG. 6 is a sectional view through an upper part of the knee joint, taken along the line B-B in FIG. 3;

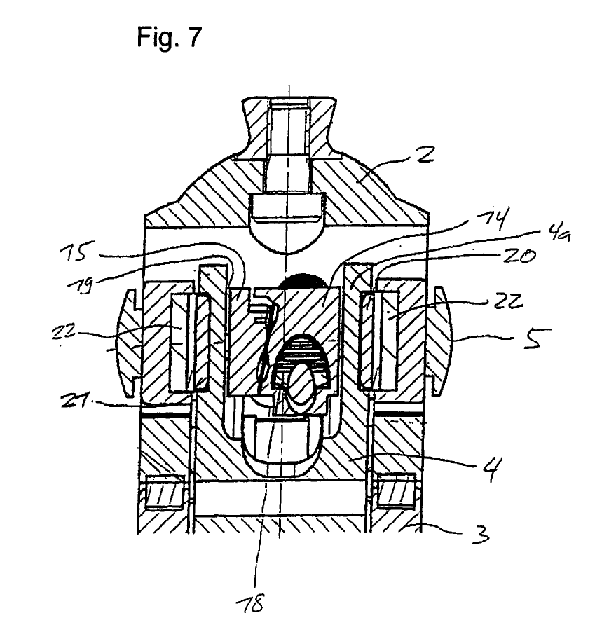

FIG. 7 is a sectional view through an upper part of the knee joint, taken along the line c-c in FIG. 3;

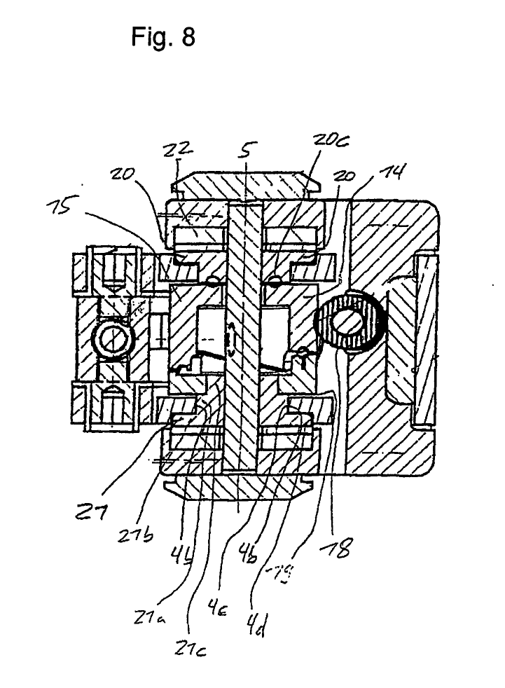

FIG. 8 is a sectional view through an upper part of the knee joint, taken along the line A-A in FIG. 3;

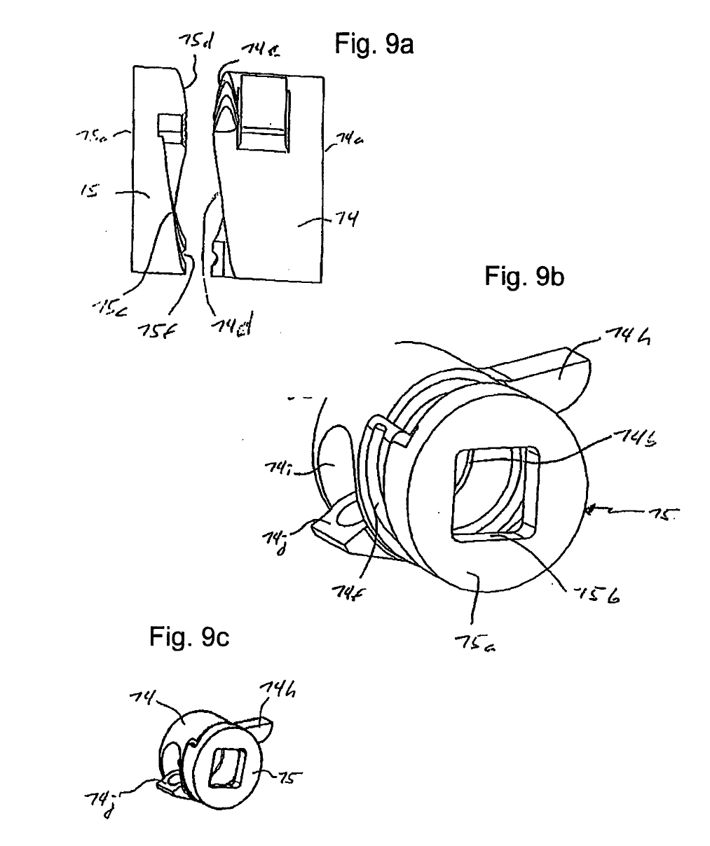

FIG. 9a is a front view of the first and the second force transmitting element of the knee joint according to an embodiment of the present invention;

FIG. 9b is a perspective view of the first and the second force transmitting element;

FIG. 9c is a perspective view of the first and the second force transmitting element in the assembled condition.

The FIGS. 1a through 1c show a knee joint, also referred to hereinafter as braking knee joint, of an embodiment of the present invention in a leg prosthesis. As can be seen from FIG. 1, said braking knee joint I has an upper joint part 2, a lower joint part 3 and an interposed central joint part 4. Said central joint part 4 is connected to the lower joint part 3 for rotation about a joint axis 5. The upper joint part 2 is connected to the central joint part 4 for pivot movement about a swing axis 6. The swing axis 6 is oriented to be parallel to the joint axis 5 and is disposed so as to be offset downward on the extension side with respect thereto.

As best shown in the FIGS. 2 through 5, the upper joint part 2 is further hinge-linked to the lower joint part 3 through a momentum phase control element 7. The momentum phase control element 7 is configured to be a piston-cylinder-system in which a piston rod 8 may be pushed into and out of a cylinder 9. Articulation occurs by fastening the piston rod 8 of the momentum phase control element 7 to the upper joint part 2 for rotation about a rotation axis 10 and the cylinder 9 of the momentum phase control element 7 to the lower joint part 3 for rotation about a rotation axis 11.

The momentum phase control element 7 is preferably configured to be a pneumatic piston-and-cylinder mechanism that dampens the movement of the piston rod 8 relative to the cylinder 9 as a function of the velocity of the movement. Alternatively, the use of a hydraulic piston-and-cylinder mechanism or of a similar device for dampening the movement of the piston rod 8 relative to the cylinder 9 may be envisaged.

In the mounted condition of the braking knee joint 1 in a leg prosthesis shown in the FIGS. 1a through 1c, the upper joint part 2 is adjoined with a thigh part 12 and the lower joint part 3, with a foot part 13. In every figure, the joint is shown in an extended condition.

The structure of the brake device of the knee joint prosthesis will be described hereinafter with reference to the FIGS. 5 through 9b. As can be seen from the FIGS. 5 and 6, a first force transmitting element 14 and a second force transmitting element 15 are disposed substantially coaxially with the joint axis 5 inside the braking knee joint.

The first force transmitting element 14 is disposed for rotation about the joint axis 5 with respect to the central part of the joint 4. The second force transmitting element 15 is restrictedly movable along the joint axis 5 but is linked to the central joint part 4 so as not to be rotatable with respect to the joint axis.

The first force transmitting element 14 and the second force transmitting element 15 are described in detail with reference to the FIGS. 9a and 9b in conjunction with FIG. 6.

As shown in the FIGS. 6 and 9a through 9c, the first force transmitting element 14 is configured to be substantially cylindrical and to have a circular coaxial hole 14b therein. The first end face 14a is configured to be circular with a substantially planar face. A coaxial circumferential groove 14e for guiding first ball bearing balls 16 is formed in the end face 14a. In the embodiment shown, the second end face 14c has three peripheral faces 14d winding upward about the coaxial hole 14b. This upward winding is such that, by extending through the circumference of the hole 14b in the counter-clockwise direction on the second end face 14c, the distance from the first end face increases until the end of the respective face 14d is reached, the distance then decreasing to the initial value in the form of a step at the beginning of the next face. On the second end face 14c, a coaxial circumferential groove 14f for guiding second ball bearing balls 17 is formed in the faces 14d.

Commencing at the cylinder jacket 14g, there is a first cam 14h on one side, said cam projecting substantially radially outward from the cylinder jacket and serving to introduce the force that will be described in detail later. On the other side of the cylinder jacket 14g there is a recess 14i substantially formed in the tangential direction and forming a second cam 14j which slightly projects from the cylinder jacket and the cam face of which serves to act on a spring element in order to adjust the actuating force.

The second force transmitting element 15 is also configured to be substantially cylindrical with a coaxial hole having a substantially square cross section. A first end face 15a of said second force transmitting element 15 is configured with a planar surface, just like the cylinder jacket surface. Similar to the second end face 14c of the first force transmitting element 14, the second end face 15c has upward winding faces 15d and a groove 15f.

FIG. 9c shows a perspective illustration of the first and of the second force transmitting element 14, 15 in the assembled condition. In this condition, the second ball bearing balls 17, which are not shown in FIG. 9c but in FIG. 6 instead, are located between the grooves 14f, 15f. As can be seen in the FIGS. 9a through 9c, the first end faces 14a, 15a are oriented to be parallel to each other. From FIG. 9c it can be seen that the distance between the first end faces 14a, 15a decreases when the second force transmitting element 15 is held stationary and the first force transmitting element 14 is rotated clockwise about the cylinder axis. Thus, the first and the second force transmitting elements 14, 15 are forming a lifting spindle.

As can be seen from FIG. 7, the central joint part 4 is configured so as to form two free legs between the two of which the first 14 and the second force transmitting element 15 are inserted in the assembled condition. Air gaps 19 are left between the first end faces 14a, 15a and the free legs 4a, 4a.

As best shown in the FIGS. 6 and 8, there are provided in the free legs 4a, 4a holes 4b, 4b that are disposed coaxially with the joint axis 5 and have, in a first portion 4c of a smaller cross section, a substantially square cross section each. In a second portion 4d, these holes each have a larger circular cross section. Brake disks 20, 21 disposed coaxially with the joint axis 5 and extending through these holes 4b, 4b are provided.

The brake disk 20 turned toward the first force transmitting element 14 has a first portion 20a with a smaller, substantially square cross section that extends perpendicular to the joint axis 5 and a second portion 20b with a larger, circular cross section. The first portion 20a is configured so as to allow movement in the hole 4b in the axial direction of the joint axis 5 but to impede rotation of the brake disk 20 about the joint axis 5 with respect to the central joint part 4. The radius of the second portion 20b is slightly smaller than the radius of the second portion 4d of the hole 4b so that it may be partially sunk thereinto. On the side turned toward the first force transmitting element 14, the brake disk 20 further has a groove 20c that is oriented coaxially with the joint axis 5 for guiding the first ball bearing balls 16.

As can be seen from the FIGS. 6 and 8, the brake disk 21 turned toward the second force transmitting element 15 also has a first portion 21a and a second portion 21b that are configured like the first 20a and the second portion 20b of the brake disk 20. The brake disk 21 however further has a third portion 21c adjoining the second portion 21b on the side turned toward the second force transmitting element 15, said third portion having a substantially square, smaller cross section than the second portion 21b. The cross section of the third portion 21c is chosen to allow engagement into the substantially square hole 15b of the second force transmitting element 15 and to secure the latter from rotating out of place with respect to the brake disk 21. Similar to the second portion 20b of the brake disk 20, the cross section of the second portion 21b is chosen to allow engagement into the substantially square hole 4b of the central joint part 4 so as to allow movement in the hole 4b in the axial direction of the joint axis 5 but to impede rotation of the brake disk 20 about the joint axis 5 with respect to the central joint part 4.

Adjacent to the side faces of the brake disks 20, 21 turned away from the force transmitting elements 14, 15, there are located disks 22 with brake lining that are preferably configured to be steel disks and are fastened in the lower joint part.

As can be seen in FIG. 5, the second cam 14j of the first force transmitting element 14 pushes against the spring element 18 the counter bearing of which is formed by a spring force set screw 23 that is connected to the upper joint part 2 through a threading. The force antagonistic to the force exerted by the spring element 18 onto the first force transmitting element 14 mounted for rotation about the joint axis 5 forms the backlash compensating screw 24 that acts onto the first cam 14h and is connected to the upper joint part 2 through a threading.

The structure described herein above allows rotation of the first force transmitting element 14 carried by the first 16 and the second ball bearing balls 17 with respect to the central joint part 4 and to the second force transmitting element 15 which is non-rotatably secured therein. Thanks to the cooperation between the first 14 and the second force transmitting element 15, which relies on the lifting spindle principle, the first end faces 14a, 15a, and through these the two brake disks 20, 21, are moved outward. The first 14 and the second force transmitting element 15 as well as the brake disks 20, 21 are thereby guided in the central joint part 4 by the joint axis 5 held in the lower joint part 3. The brake disks 20, 21, which are moved outward, push against the disks 22 fastened in the lower joint part 3, thus braking a movement of the central joint part 4 with respect to the lower joint part 3 about the joint axis 5.

With reference to FIG. 3, bending movement of the braking knee joint 1 will now be described without actuation of the braking device.

When the braking knee joint is slowly bent with little force, the central joint part 4 is rotated clockwise about the joint axis 5 with respect to the lower joint part 3. Upon this rotation, the swing axis 6 also moves clockwise about the joint axis 5. Accordingly, the swing axis moves on a circular path upward toward the left in FIG. 3. When the exerted force is low, the upper joint part 2 coupled on the central joint part 4 to the swing axis 6 moves also clockwise together with central joint part 4 so that the rotation axis 10 also moves clockwise on a circular path about the joint axis 5. Accordingly, the rotation axis 10 moves substantially downward in FIG. 3.

Upon downward movement of the rotation axis 10, the piston rod 8 is pushed into the cylinder 9 and the momentum phase control element 7 slightly rotates about the rotation axes 10 and 11 relative to the upper joint part 2 and to the lower joint part 13.

Upon extension of the braking knee joint, the movement described herein above occurs in reverse.

Referring to FIG. 5, the actuation of the braking device by loading the foot will be described hereinafter.

A predetermined actuation force is set via the spring element 18 by means of the spring force set screw 23. This force is adjusted as a function of the weight and the activity of the prosthesis wearer.

When no vertical force is acting from the top onto the upper joint part 2 and when the spring element 18 is pre-biased, the first cam 14h pushes the edge 2a of the upper joint part 2, which is rotatable about the swing axis 6 with respect to the central joint part 4, against the limit stop 4a of the central joint part 4. In this situation, the first 14 and the second force transmitting element 15 are in a rest condition in which the brake disks 20, 21 are not pushed outward so that rotation of the central joint part 4 about the joint axis 5 relative to the lower joint part 3 is not braked.

If a vertical force component acts from the top onto the upper joint part 2 in such a manner that the torque transmitted by the backlash compensating screw 24 through the first cam 14h onto the first force transmitting element 14 exceeds the torque transmitted by the spring element 18 through the second cam 14j, the upper joint part 2 tilts about the swing axis 6 with respect to the central joint part 4. Upon tilting, the first force transmitting element 14 is caused to rotate about the joint axis 5 with respect to the second force transmitting element 15 so that the brake disks 20, 21 are moved outward as already described herein above and rotation of the central joint part 4 about the joint axis 5 relative to the lower joint part 3 is braked.

If the vertical force component is removed, the Belleville spring washer 18 pushes the first force transmitting element 14 back and, as a result thereof, the upper joint part 2 is pushed back into the limit stop 4a provided on the central joint part 4 so that the brake is allowed to come free.

The actuation force of the disk brake can be adjusted through the spring force set screw 23, either increasing or decreasing the pretension of the Belleville spring washer 18 by turning the spring force set screw 23.

The first force transmitting element 14 can be rotated forward with respect to the second force transmitting element 15 using the backlash compensating screw 24 so that readjustment can readily occur upon wear of the brake linings.

As can be seen in the FIGS. 1a through 1c, the knee joint prosthesis described herein above has, due to the particular arrangement of the swing axis 6, which is disposed parallel on the extension side and offset downward with respect to the joint axis 5 on the side of the lower joint part, the particular advantage that the braking effect of the disk brake depends on which phase of a step the leg prosthesis is performing.

In FIG. 1a it can be seen that, with respect to a force acting vertically from the top, the swing axis 6 forms a great lever B with respect to the joint axis 5 when the heel of the leg prosthesis is placed on the floor, i.e., the braking effect is operative when the leg prosthesis is subjected to quite small a weight load. As shown in FIG. 1c, the lever A formed when the point of the foot is placed on the floor is much smaller so that the body load onto the leg prosthesis must be higher in order to achieve the braking effect, meaning that the brake releases sooner upon unloading. In particular in connection with the disk brake, this effect allows for a more natural movement of the leg during walking since the brake is prevented from still being engaged when the momentum phase of the leg is being initiated. Concurrently, high stability of the leg prosthesis is achieved in the standing phase.

The use of the disk brake leads to significant reduction of wear in the brake device so that the maintenance intervals may be reduced and the reliability of the knee joint prosthesis may be significantly improved.

The implementation of the knee joint prosthesis with the two force transmitting elements 14, 15 further involves that the magnitude of the braking effect may be controlled by the prosthesis wearer dosing the exerted vertical force component so that the properties of the knee joint prosthesis are significantly improved. When used in combination with an air-operated momentum phase control element 7, the weight of the knee joint prosthesis may be kept low, thus increasing wearing comfort.

It is also contemplated to e.g., configure the first force transmitting element in the shape of a wedge that is urged between two inclined planes when the actuating force is exerted, causing these to be pushed outward in order to exert the force onto at least onr of the brake disks. With such an apparatus, the wedge is ejected when the load is removed so that a braking force is no longer exerted onto the brake disks.

Likewise, it is contemplated to realise the force transmission through a bell-crank lever system. It may also be envisaged to realise the force transmission through a hydraulic apparatus in which the actuating force acts onto a piston and is transmitted to a second piston which then exerts the transmitted force onto the brake disk.

1. A braking knee joint for a leg prosthesis with an upper joint part (2) and a lower joint part (3) rotatable about a joint axis (5) relative to said upper joint part (3), and with a braking device (14, 15, 20, 21), characterized by a first force transmitting element (14) onto which a force producing the braking effect is acting and which transfers at a prescribed transmission ratio said force onto a second force transmitting element (15) which exerts the transferred force onto a brake disk (20, 21) in a direction substantially parallel with respect to the joint axis (5).

2. The braking knee joint as set forth in claim 1, wherein the first and the second force transmitting element (14, 15) form an inclined plane for transferring the force.

3. The braking knee joint as set forth in claim 1, with a central joint part (4) provided between the upper joint part (2) and the lower joint part (3), said central joint part (4) being connected to either the upper joint part (2) or the lower joint part (3) through the joint axis (5) and to the respective other one through a swing axis (6).

4. The braking knee joint as set forth in claim 3, wherein the swing axis (6) is disposed on the extension side, parallel with respect to the joint axis (5) and on the side of the lower joint part (3).

5. The braking knee joint as set forth in claim 1, wherein the first and the second force transmitting element (14, 15) are configured so that the first force transmitting element (14) is caused to rotate about a rotation axis which is substantially parallel to the joint axis (5) by the force producing the braking effect and that the transferred force causes the second force transmitting element (15) to perform a translation substantially parallel to the joint axis (5).

6. The braking knee joint as set forth in claim 1, wherein the first force transmitting element (14) and the second force transmitting element (15) form a lifting spindle disposed on the joint axis (5).

7. The braking knee joint as set forth in claim 1, wherein the force transmission occurs through a hydraulic system.

8. The braking knee joint as set forth in claim 1, wherein the force transmission occurs through a bell-crank lever.

9. The braking knee joint as set forth in claim 1, wherein the first or the second force transmitting element is configured in the shape of a wedge.

10. The braking knee joint as set forth in claim 1, wherein brake disks (20, 21) are provided on either side of the second force transmitting element (15) in the direction of the joint axis (5).

11. The braking knee joint as set forth in claim 1, wherein the pre-adjustment of the force producing the braking effect is achieved by a pre-biased spring (18).

12. The braking knee joint as set forth in claim 1, wherein a piston-and-cylinder device (7) is provided for damping walking movement.

13. The braking knee joint as set forth in claim 1, wherein the braking device (14, 15, 20, 21) is actuatable by loading the foot.

14. The braking knee joint as set forth in claim 1, wherein the force acting onto the first force transmitting element (14) acts substantially at right angles with respect to the joint axis.
